(19) United States
(12) Patent Application Publication
(10) Pub. No.: US 2026/0325785 A1
(43) Pub. Date: Oct. 22, 2026

(54) SYSTEM AND METHOD FOR PERFORMING AN ELECTROENCEPHALOGRAPHY (EEG) ANALYSIS AND A PROGNOSIS MEASUREMENT

(71) Applicant: Andrew Magdy Kamal, Washington Township, MI (US)

(72) Inventor: Andrew Magdy Kamal, Washington Township, MI (US)

(21) Appl. No.: 19/183,801

(22) Filed: Apr. 19, 2025

(51) Int. Cl. A61M 21/00

(52) U.S. Cl. CPC A61M 21/00; A61M 2021/0027

(57) ABSTRACT

The present disclosure provides a system and a method for performing an Electroencephalography (EEG) diagnostics and a prognosis measurement, which includes determining one or more parameters of an EEG frequency waveform captured during the EEG diagnostics. The determined one or more parameters of the EEG frequency waveform are evaluated prior to applying binaural beats or acoustic amplification to the EEG frequency waveform. Further, the evaluated one or more parameters of the EEG frequency waveform and one or more parameters of a frequency waveform corresponding to a correlating diagnosis signal are compared. The comparison is performed using a core metric and is done to perform the prognosis measurement.

### PRIORITY STATEMENT

The present application hereby claims priority under 35 U.S.C. § 119(e) to U.S. provisional patent application No. 63/639,586 filed on Apr. 27, 2024, the entire contents of which are hereby incorporated herein by reference.

### TECHNICAL FIELD

The embodiments of the present disclosure generally relate to a field of diagnosis and analysis of mental disorders, and specifically to a system and a method for performing an Electroencephalography (EEG) diagnostics and a prognosis measurement.

### BACKGROUND OF INVENTION

The following description of the related art is intended to provide background information pertaining to the field of the disclosure. This section may include certain aspects of the art that may be related to various features of the present disclosure. However, it should be appreciated that this section is used only to enhance the understanding of the reader with respect to the present disclosure, and not as admissions of the prior art.

Typically, a non-invasive Electroencephalography (EEG) system is used to measure an electrical activity of a human brain. The EEG system acquires various signals by placing electrodes over a human scalp and records voltage variations that are generated during neural communications. A conventional EEG system has mainly 14 to 64 electrodes that are linked to an amplifier and facilitates formation of a multidimensional signal.

Though features of the EEG system such as user-friendliness, safety, and high temporal resolution makes it ideal for various tasks, there is still a limitation observed with an optimum utilization of available EEG headsets and external Brain Computer Interfaces (BCIs) to obtain results to be used for prognosis measurements.

There is, therefore, a need in the art for an improved system and method that facilitates to provide an accurate diagnostic using the EEG system, a prognosis computation assessment and suggestion for a relevant therapy.

### OBJECTS OF THE INVENTION

Some of the objects of the present disclosure, which at least one embodiment herein satisfies are listed herein below.

It is an object of the present disclosure to provide a system and a method to perform an Electroencephalography (EEG) diagnostics and a prognosis measurement.

It is an object of the present disclosure to provide a system and a method that analyzes wavelength height frequencies for certain disorder types by utilizing EEG scans, signal topology of the EEG scans, and raw data.

It is an object of the present disclosure to provide a system and a method that sends signals through an EEG headset or an external non-implantable Brain-Computer-Interface (BCI).

It is an object of the present disclosure to provide a system and a method that enables sending signals by utilizing binaural beats within a 30 HZ range or less to the brain to follow a neural interfacing or EEG frequency range of wavelengths.

Yet another object of the present disclosure is to provide a system and a method that analyzes EEG scan data by using a mathematical proof for a base data of EEG parameters of a healthy individual versus EEG parameters of a patient.

### SUMMARY

This section is provided to introduce certain objects and aspects of the present disclosure in a simplified form that are further described below in the detailed description. This summary is not intended to identify the key features or the scope of the claimed subject matter.

In an aspect, the present disclosure relates to a system for performing an Electroencephalography (EEG) diagnostics and a prognostic measurement. The system includes one or more processors and a memory operatively coupled to the one or more processors, wherein the memory comprises processor-executable instructions, which on execution may cause the one or more processors to determine one or more parameters of an EEG frequency waveform captured during the EEG diagnostics. The one or more processors may evaluate the determined one or more parameters of the EEG frequency waveform prior to applying binaural beats or acoustic amplification to the EEG frequency waveform. Further, the one or more processors may compare the evaluated one or more parameters of the EEG frequency waveform and one or more parameters of a frequency waveform corresponding to a correlating diagnosis signal to perform the prognostic measurement. The comparison may be performed using a core metric.

In an embodiment, the one or more processors may use a pre-determined internal signal topology to perform the prognosis measurement and recommend a remedial treatment.

In an embodiment, the evaluated one or more parameters of the EEG frequency waveform may be any or a combination of height, length, and frequency of the EEG frequency waveform, and the one or more parameters of the frequency waveform corresponding to the correlating diagnosis signal may be any or a combination of height, length, and frequency of the waveform corresponding to the correlating diagnosis signal.

In an embodiment, the core metric may be computed based on any or a combination of the height of the EEG frequency waveform, a minimal height of the frequency waveform of the correlating diagnosis signal, an endpoint of the frequency of the EEG frequency waveform, and a top point of the frequency of the EEG frequency waveform.

In an embodiment, a base of the height of the EEG frequency waveform and the minimal height of the frequency waveform of the correlating diagnosis signal may be horizontally scalable.

In an embodiment, the EEG diagnostics may be performed using an EEG headset and an external non-invasive Brain-Computer Interface (BCI), and the prognostic measurement may be performed using one or more deep brain simulation mechanisms.

In an embodiment, the EEG headset may be any of a dry node EEG headset and a wet node EEG headset.

In an embodiment, the EEG diagnostics and the prognostic measurement may be performed in real-time without data latency and signal sensitivity.

In an embodiment, each of the EEG frequency waveform and the waveform corresponding to the correlating diagnosis signal may be simulated within an EEG simulator to be used in clinical trials.

In an embodiment, the EEG frequency waveform may be implemented in any of an EEG power spectrum, one or more spectral frequency ranges, a spectrum analysis, a magnitudal activity, and an awake/non awake EEG spectral, neuronal and density.

In an embodiment, a modular form of Binaural Beat Therapy (BBT) may be derived from the EEG frequency waveform. The BBT may be used to determine one or more base data points to perform a therapeutic neurostimulation.

In an embodiment, an Exposure and Response Prevention (ERP) therapy may be used for the prognostic measurement without triggering recessive brain or neural patterns.

In an embodiment, a geometric topology may be applied into a wave signal analysis in relation to raw EEG data along with one or more signal readings.

In an embodiment, the one or more parameters of the EEG frequency waveform of the diagnosis signal may be modulated, triggered, and oscillated to perform the EEG diagnostics.

In an aspect, the present disclosure relates to a method for performing an EEG diagnostics and a prognostic measurement. The method includes determining, by one or more processors, one or more parameters of an EEG frequency waveform captured during the EEG diagnostics. Further, the method includes evaluating the determined one or more parameters of the EEG frequency waveform prior to applying binaural beats or acoustic amplification to the EEG frequency waveform. In addition, the method includes comparing the evaluated one or more parameters of the EEG frequency waveform and one or more parameters of a frequency waveform corresponding to a correlating diagnosis signal to perform the prognostic measurement. The comparison may be performed using a core metric.

### BRIEF DESCRIPTION OF THE DRAWINGS

The accompanying drawings, which are incorporated herein, and constitute a part of this disclosure, illustrate exemplary embodiments of the disclosed methods and systems which like reference numerals refer to the same parts throughout the different drawings. Components in the drawings are not necessarily to scale, emphasis instead being placed upon clearly illustrating the principles of the present disclosure. Some drawings may indicate the components using block diagrams and may not represent the internal circuitry of each component. It will be appreciated by those skilled in the art that disclosure of such drawings includes the disclosure of electrical components, electronic components, or circuitry commonly used to implement such components.

The diagrams are for illustration only, which thus is not a limitation of the present disclosure, and wherein:

FIG. 1 illustrates an exemplary block diagram of a diagnostic and prognosis system, in accordance with an embodiment of the present disclosure.

FIG. 2 illustrates an exemplary flowchart representation for performing an EEG diagnostics and a prognosis measurement, in accordance with an embodiment of the disclosure.

FIG. 3 illustrates an exemplary flowchart representation for performing an EEG frequency waveform analysis, in accordance with an embodiment of the disclosure.

FIG. 4 illustrates an exemplary flowchart representation for analyzing a normal beta waveform and an abnormal beta waveform along with creating a core metric, in accordance with embodiments of the disclosure.

FIG. 5A illustrates an exemplary graph representing height and length metrics over time, in accordance with embodiments of the disclosure.

FIG. 5B illustrates an exemplary graph representing an abnormal height and length metrics over time, in accordance with embodiments of the disclosure.

FIG. 6 illustrates a sequence flow for performing the EEG diagnostics and the prognostic measurement, in accordance with an embodiment of the present disclosure.

FIG. 7 illustrates an exemplary computer system in which or with which embodiments of the present disclosure may be utilized in accordance with embodiments of the present disclosure.

The foregoing shall be more apparent from the following more detailed description of the invention.

### DETAILED DESCRIPTION

In the following description, for the purposes of explanation, various specific details are set forth in order to provide a thorough understanding of embodiments of the present disclosure. It will be apparent, however, that embodiments of the present disclosure may be practiced without these specific details. Several features described hereafter can each be used independently of one another or with any combination of other features. An individual feature may not address all of the problems discussed above or might address only some of the problems discussed above. Some of the problems discussed above might not be fully addressed by any of the features described herein.

The ensuing description provides exemplary embodiments only, and is not intended to limit the scope, applicability, or configuration of the disclosure. Rather, the ensuing description of the exemplary embodiments will provide those skilled in the art with an enabling description for implementing an exemplary embodiment. It should be understood that, various changes may be made in the function and arrangement of elements without departing from the spirit and scope of the invention as set forth.

Specific details are given in the following description to provide a thorough understanding of the embodiments. However, it will be understood by one of ordinary skill in the art that the embodiments may be practiced without these specific details. For example, circuits, systems, networks, processes, and other components may be shown as components in block diagram form in order not to obscure the embodiments in unnecessary detail. In other instances, well-known circuits, processes, algorithms, structures, and techniques may be shown without unnecessary detail in order to avoid obscuring the embodiments.

Also, it is noted that individual embodiments may be described as a process which is depicted as a flowchart, a flow diagram, a data flow diagram, a structure diagram, or a block diagram. Although a flowchart may describe the operations as a sequential process, many of the operations can be performed in parallel or concurrently. In addition, the order of the operations may be re-arranged. A process is terminated when its operations are completed but could have additional steps not included in a figure. A process may correspond to a method, a function, a procedure, a subroutine, a subprogram, etc. When a process corresponds to a function, its termination can correspond to a return of the function to the calling function or the main function.

The word “exemplary” and/or “demonstrative” is used herein to mean serving as an example, instance, or illustration. For the avoidance of doubt, the subject matter disclosed herein is not limited by such examples. In addition, any aspect or design described herein as “exemplary” and/or “demonstrative” is not necessarily to be construed as preferred or advantageous over other aspects or designs, nor is it meant to preclude equivalent exemplary structures and techniques known to those of ordinary skill in the art. Furthermore, to the extent that the terms “includes,” “has,” “contains,” and other similar words are used in either the detailed description or the claims, such terms are intended to be inclusive—in a manner similar to the term “comprising” as an open transition word-without precluding any additional or other elements.

As used herein, “connect”, “configure”, “couple” and its cognate terms, such as “connects”, “connected”, “configured”, and “coupled” may include a physical connection (such as a wired/wireless connection), a logical connection (such as through logical gates of semiconducting device), other suitable connections, or a combination of such connections, as may be obvious to a skilled person.

As used herein, “send”, “transfer”, “transmit”, and their cognate terms like “sending”, “sent”, “transferring”, “transmitting”, “transferred”, “transmitted”, etc. include sending or transporting data or information from one unit or component to another unit or component, wherein the content may or may not be modified before or after sending, transferring, transmitting.

Reference throughout this specification to “one embodiment” or “an embodiment” or “an instance” or “one instance” means that a particular feature, structure, or characteristic described in connection with the embodiment is included in at least one embodiment of the present invention. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

The terminology used herein is for the purpose of describing particular embodiments only and is not intended to be limiting of the invention. As used herein, the singular forms “a,” “an,” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items.

The present disclosure relates to a system and a method for performing an Electroencephalography (EEGs) diagnostics, analysis and a prognosis determination for assessment of a human brain and recommending a corresponding therapy. From a mathematical analysis standpoint, the disclosed system and method indicates using a correct signal topology with an appropriate comparison metric for achieving a reference point. The disclosed system and method represent a mathematical proof and a computational algorithm that modifies a core hardware utilization, which is a feature that is not present in any of the available EEG headsets or the external Brain Computer Interfaces BCIs currently available in the market.

The disclosed system and method involve analyzing wavelength height frequencies that are specific to certain disorder types by using EEG scans and their signal topology alongside raw data. To ensure accuracy and to eliminate conventional bias, the disclosed system and method considers mood of a patient during the EEG scans. While performing the EEG scans, signals below 30 Hz are transmitted using binaural beats to the brain through the EEG headset or the BCI. This encourages the binaural beats to align with a neural interfacing or an EEG frequency range corresponding to a specific disorder's wavelength height topology. The disclosed system and method may serve as a form of an Exposure and Response Prevention (ERP) therapy or a more ethical, non-invasive alternative to some types of deep brain stimulation mechanisms when performed computationally by reading the patient's EEG and transmitting relevant signals.

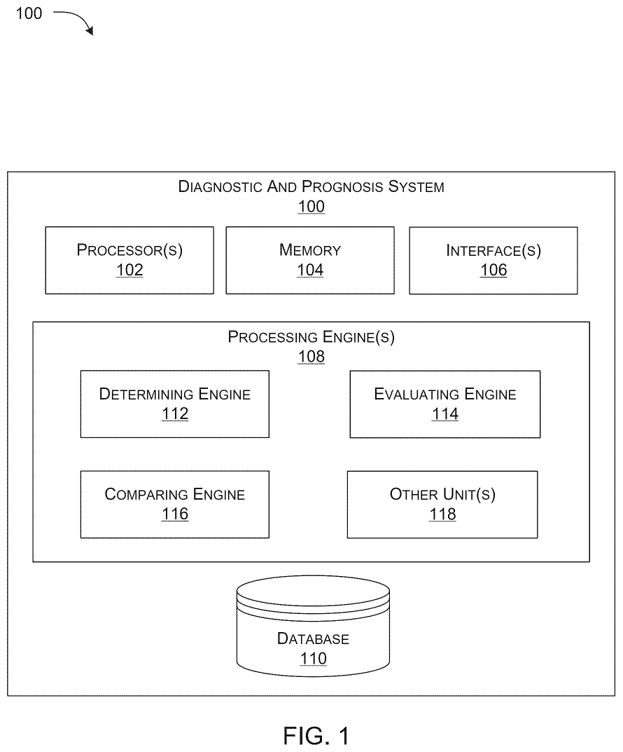

FIG. 1 illustrates an exemplary block diagram of a diagnostic and prognosis system 100, in accordance with an embodiment of the present disclosure. The diagnostic and prognosis system 100 may also be referred to as a system 100 herewith henceforth. The system 100 may include one or more processors 102. The one or more processors 102 may be implemented as one or more microprocessors, microcomputers, microcontrollers, digital signal processors, central processing units, logic circuitries, and/or any devices that manipulate data based on operational instructions. Among other capabilities, the one or more processors 102 may be configured to fetch and execute computer-readable instructions stored in a memory 104 of the system 100. The memory 104 may store one or more computer-readable instructions or routines, which may be fetched and executed to create or share the data units over a network service. The memory 104 may include any non-transitory storage device including, for example, volatile memory such as Random-Access Memory (RAM), or non-volatile memory such as an Erasable Programmable Read-Only Memory (EPROM), a flash memory, and the like.

In an embodiment, the system 100 may also include an interface(s) 106. The interface(s) 106 may include a variety of interfaces, for example, a variety of interfaces, for example, interfaces for data input and output devices, referred to as I/O devices, storage devices, and the like. The interface(s) 106 may facilitate communication of the system 100 with various devices coupled to it. The interface(s) 106 may also provide a communication pathway for one or more components of the system 100. Examples of such components include, but are not limited to, processing engine(s) 108 and a database 110.

In an embodiment, the processing engine(s) 108 may be implemented as a combination of hardware and programming (for example, programmable instructions) to implement one or more functionalities of the processing engine(s) 108. In examples, described herein, such combinations of hardware and programming may be implemented in several different ways. For example, the programming for the processing engine(s) 108 may be processor-executable instructions stored on a non-transitory machine-readable storage medium and the hardware for the one or more processors 102 may include a processing resource, to execute such instructions. In the present examples, the machine-readable storage medium may store instructions that, when executed by the processing resource, implement the processing engine(s) 108. In such examples, the system 100 may comprise the machine-readable storage medium storing the instructions and the processing resource to execute the instructions, or the machine-readable storage medium may be separate but accessible to the system 100 and the processing resource. In other examples, the processing engine(s) 108 may be implemented by an electronic circuitry.

In an embodiment, the database 110 may comprise data that may be either stored or generated as a result of functionalities implemented by any of the components of the processors 102 or the processing engine(s) 108 or the system 100.

In an exemplary embodiment, the processing engine(s) 108 may include one or more engines selected from any of a determining engine 112, an evaluating engine 114, a comparing engine 116 and other units/engines 118. The other units/engines 118 may include, but are not limited to, a monitoring engine, a determination engine, and the like.

In an embodiment, the one or more processors 102 may via the determining engine 112 determine one or more parameters of an EEG frequency waveform that are captured during an EEG diagnostics. The EEG frequency waveform is interchangeably also referred to as the EEG frequency wave herewith.

In an embodiment, the evaluating engine 114 may evaluate the determined one or more parameters of the EEG frequency waveform prior to applying binaural beats or acoustic amplification to the EEG frequency waveform. The evaluated one or more parameters of the EEG frequency waveform may include one or more of height, length, and frequency of the EEG frequency waveform.

In an embodiment, the comparing engine 116 may compare the evaluated one or more parameters of the EEG frequency waveform and one or more parameters of a frequency waveform corresponding to a correlating diagnosis signal to perform a prognosis measurement. The one or more parameters of the frequency waveform corresponding to the correlating diagnosis signal may be any or a combination of height, length, and frequency of the waveform corresponding to the correlating diagnosis signal. It is to be noted that the correlating diagnosis signal represents a distorted waveform and may be used for the prognostic measurement and for determining a reference disease type.

The comparison may be performed using a core metrics that is computed based on any or a combination of the height of the EEG frequency waveform, a minimal height of a frequency waveform of the correlating diagnosis signal, an endpoint of the frequency of the EEG frequency waveform, and a top point of the frequency of the EEG frequency waveform. A base of the height of the EEG frequency waveform and the minimal height of the frequency waveform of the correlating diagnosis signal may be horizontally scalable.

Further, the one or more parameters of the EEG frequency waveform of the diagnosis signal may be modulated, triggered, and oscillated to perform the EEG diagnostics. In addition, each of the EEG frequency waveform and the waveform corresponding to the correlating diagnosis signal may be simulated within an EEG simulator to be used in clinical trials.

In an embodiment, a pre-determined internal signal topology may be used to perform the prognosis measurement and recommend a remedial treatment. The EEG diagnostics may be performed by using any of the EEG headset, the external non-invasive BCI. The EEG headset may be any of a dry node EEG headset and a wet node EEG headset. Further, the prognosis measurement may be performed using deep brain simulation mechanisms. It is to be appreciated that the EEG diagnostics and the prognosis measurement may be performed in real-time without data latency and signal sensitivity.

Further, the EEG frequency waveform may be implemented in any of an EEG power spectrum, one or more spectral frequency ranges, a spectrum analysis, a magnitudal activity, and an awake/non awake EEG spectral, neuronal and density.

A modular form of Binaural Beat Therapy (BBT) may be derived from the EEG frequency waveform. The BBT may be used to determine one or more base data points to perform a therapeutic neurostimulation.

In an embodiment, the ERP therapy may be used for the prognosis measurement without triggering recessive brain or neural patterns. Further, a geometric topology may be applied into a wave signal analysis in relation to raw EEG data along with one or more signal readings.

Although FIG. 1 shows exemplary components of the system 100, in other embodiments, the system 100 may include fewer components, different components, differently arranged components, or additional functional components than depicted in FIG. 1. Additionally, or alternatively, one or more components of the system 100 may perform functions described as being performed by one or more other components of the system 100.

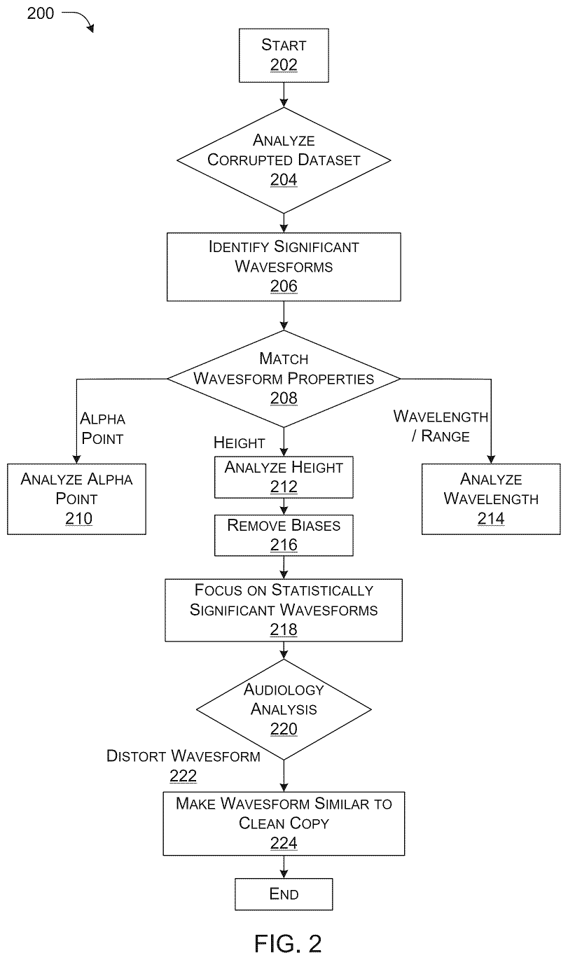

FIG. 2 illustrates an exemplary flowchart representation 200 for performing the EEG diagnostics and the prognosis measurement, in accordance with an embodiment of the disclosure. The EEG scan is typically used for diagnosing and analyzing various mental and psychological disorders and disabilities, such as parkinson's, autism spectrum disorder, Obsessive-Compulsive Disorder (OCD), and depression among others. The disclosed system and method analyzes wavelength height frequencies specific to a certain disorder type using the EEG scans and a signal topology alongside raw data.

With respect to FIG. 2, at step 202, prior to initiating the diagnosis and the measurement, a clean copy dataset may be taken into consideration. The clean copy dataset may look like a normal EEG scan. At step 204, a corrupted dataset may be analyzed to obtain a scanned data of an abnormal EEG scan. Next, at step 206, multiple significant waveforms may be identified by matching one or more properties of a waveform at step 208. Each of multiple significant waveforms may be analyzed on basis of an alpha point (length) at step 210, height at step 212, and wavelength/range (frequency) at step 214. On the basis of the analyzed height, length and frequency of each of the multiple significant waveforms, a disorder may be diagnosed. In an embodiment, biases in the waveform calculations may be affected by mood. The biases may be eliminated at step 216. Once complete non-bias statistically significant waveforms are obtained at step 218, an audio may be sent for audiology analysis at step 220 to distort the waveforms at step 222. This step may be performed to make the bias waveforms similar to the non-bias waveforms, at step 224.

Subsequently, through the EEG headset or the external non-implantable BCI, one or more signals may be transmitted using binaural beats below 30 Hz to the brain. This may encourage the signals to align with a neural interfacing or the EEG frequency waveform range corresponding to a specific disorder's wavelength height topology.

The disclosed system may serve as a form of the ERP therapy or a more ethical, non-invasive alternative to some types of deep brain stimulation mechanisms, when the EEG scans are performed computationally by reading the patient's EEG frequency waveform and accordingly transmitting signals. The following equations implement a form of an internal signal topology:

$${base}_{y} \vert diff ( x ) * average {\sum }_{\vec{AB}} / Δ x \qquad eq ( 1 )$$

$$or$$

$${base}_{y} \vert diff ( x ) * average \frac{Σ}{\vec{ab}} / Δ x \qquad eq ( 2 )$$

where it is assumed that y represents a regular signal EEG frequency wave height, x represents a minimal height of a wave related to a correlating diagnosis, A represents an end of a point of a frequency, and B represents a top point of the same frequency. This is then divided by a change (delta) of x.

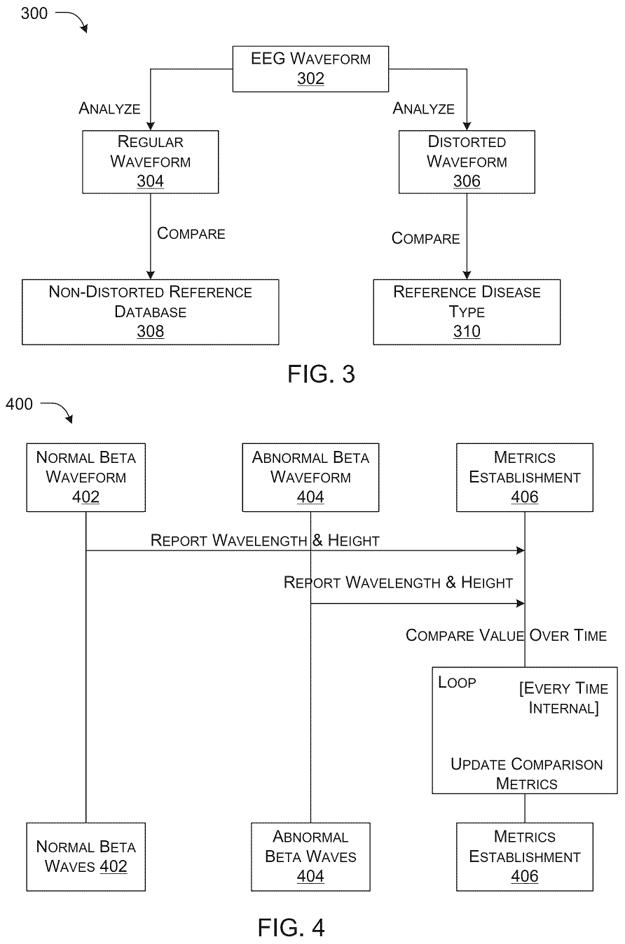

FIG. 3 illustrates an exemplary flowchart representation 300 for performing an EEG frequency waveform analysis, in accordance with an embodiment of the disclosure. As is illustrated, the EEG frequency waveform 302 may be analyzed as a regular waveform, at 304. The regular waveform may be compared to a non-distorted waveform available at a reference database, at 308. Further, the EEG frequency waveform may be analyzed as a distorted waveform, at 306. The distorted waveform represents the frequency waveform corresponding to the correlating diagnosis signal. The distorted waveform may be compared to provide a prognostic measurement and determine a reference disease type, at 310.

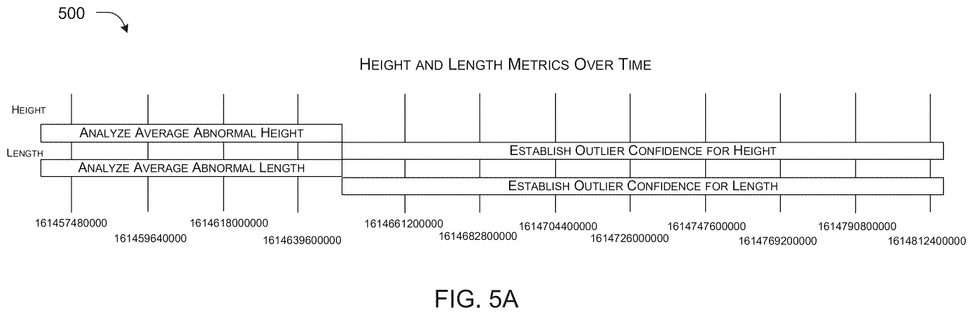

FIG. 4 illustrates an exemplary flowchart representation 400 for analyzing a normal beta waveform 402 and an abnormal beta waveform 404 along with creating a core metric 406, in accordance with embodiments of the disclosure. As is illustrated, both the normal beta waveform 402 and the abnormal beta waveform 404 may be analyzed to report their respective wavelength information and height information. The wavelength and the height information may be used for establishing the core metric 406. Further, the wavelength and the height information of each of the normal beta waveform 402 and the abnormal beta waveform 404 may be compared over time for a fixed time interval to update a comparison metric and for creating the correct core metric 406.

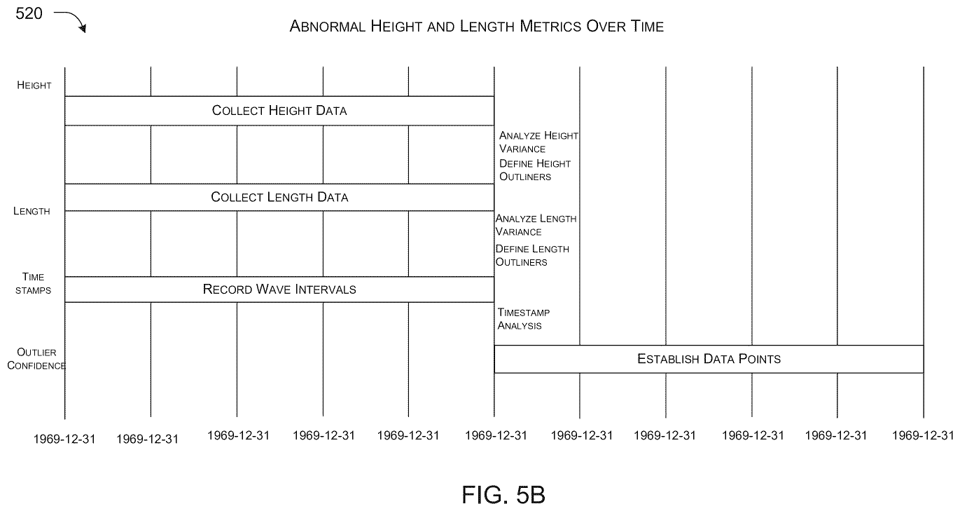

FIG. 5A illustrates an exemplary graph 500 representing a height and length metrics over time, in accordance with embodiments of the disclosure.

FIG. 5B illustrates an exemplary graph 520 representing an abnormal height and length metrics over time, in accordance with embodiments of the disclosure.

The disclosed system and method uses a Lang's emotional dimension classificational model to consider emotional states for cognitive biases. It may be appreciated that other alternative available emotional state models may as well be used. Emotions may include categories within, for example, love types (eroticism and platonic), relaxed, bored, happy, excited, depressed, sad, fear, and hate.

In an embodiment, an EEG scanning may be performed on the patient for about 48 to 72 hours to obtain a standard base dataset. The EEG scan data may be analyzed by utilizing a mathematical proof for a base data of the one or more parameters of the EEG frequency waveform of a healthy individual versus the one or more parameters of a EEG frequency waveform of the patient.

In an embodiment, an interval signal topology may be defined as in eq (1):

$${base}_{y} \vert diff ( x ) * average {\sum }_{\vec{AB}} / Δ x \qquad eq ( 1 )$$

In an embodiment, ranges of the EEG frequency waveform may be implemented in relation to a frequency differentiation to send audiovisual or external signals (binaural beats) to a human brain for a few hours, so that the brain subscribes to a particular pattern. When focused on reprogramming on a subconscious level, time ranges may be changed depending on the diagnosis.

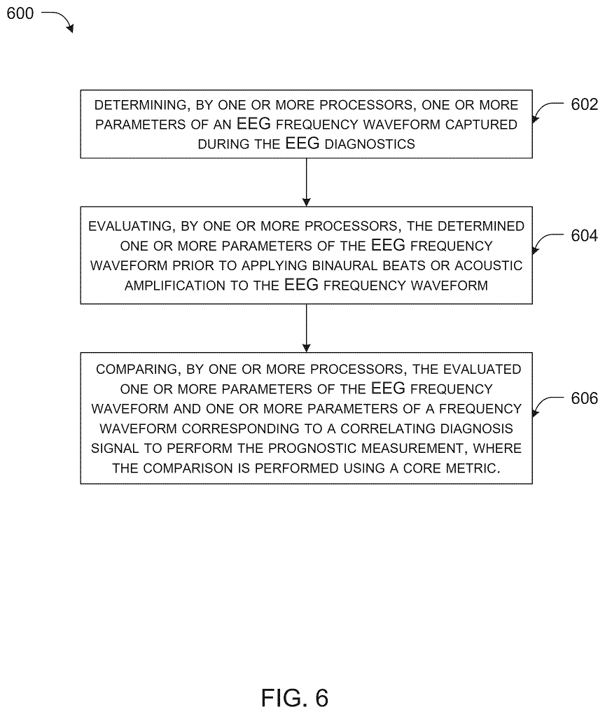

FIG. 6 illustrates a sequence flow 600 for performing the EEG diagnostics and the prognostic measurement, in accordance with an embodiment of the present disclosure. With reference to FIG. 6, the method 600 may include the following steps. At step 602, one or more parameters of the EEG frequency waveform captured during the EEG diagnostics may be determined. At step 604, the determined one or more parameters of the EEG frequency waveform may be evaluated prior to applying binaural beats or acoustic amplification to the EEG frequency waveform. Further, at step 606, the evaluated one or more parameters of the EEG frequency waveform and one or more parameters of a frequency waveform corresponding to a correlating diagnosis signal may be compared to perform the prognosis measurement. The comparison may be performed using a core metric.

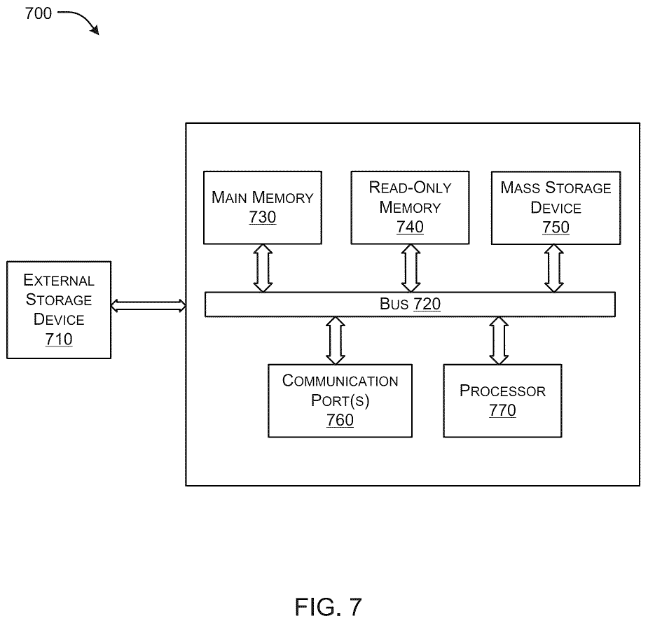

FIG. 7 illustrates an exemplary computer system 700 in which or with which embodiments of the present disclosure may be utilized in accordance with embodiments of the present disclosure.

As shown in FIG. 7, the computer system 700 may include an external storage device 710, a bus 720, a main memory 730, a read-only memory 740, a mass storage device 750, a communication port(s) 760, and a processor 770. A person skilled in the art will appreciate that the computer system 700 may include more than one processor 770 and communication ports 760. The processor 770 may include various modules associated with embodiments of the present disclosure. The communication port(s) 760 may be any of an RS-232 port for use with a modem-based dialup connection, a 10/100 Ethernet port, a Gigabit or 10 Gigabit port using copper or fiber, a serial port, a parallel port, or other existing or future ports. The communication ports(s) 760 may be chosen depending on a network, such as a Local Area Network (LAN), Wide Area Network (WAN), or any network to which the computer system 700 connects.

In an embodiment, the main memory 730 may be a Random-Access Memory (RAM), or any other dynamic storage device commonly known in the art. The read-only memory 740 may be any static storage device(s) e.g., but not limited to, a Programmable Read Only Memory (PROM) chip for storing static information e.g., start-up or basic input/output system (BIOS) instructions for the processor 770. The mass storage device 750 may be any current or future mass storage solution, which can be used to store information and/or instructions. Exemplary mass storage solutions include, but are not limited to, Parallel Advanced Technology Attachment (PATA) or Serial Advanced Technology Attachment (SATA) hard disk drives or solid-state drives (internal or external, e.g., having Universal Serial Bus (USB) and/or Firewire interfaces).

In an embodiment, the bus 720 may communicatively couple the processor(s) 770 with the other memory, storage, and communication blocks. The bus 720 may be, e.g., a Peripheral Component Interconnect (PCI)/PCI Extended (PCI-X) bus, Small Computer System Interface (SCSI), USB, or the like, for connecting expansion cards, drives, and other subsystems as well as other buses, such a front side bus (FSB), which connects the processor 770 to the computer system 700.

In another embodiment, operator and administrative interfaces, e.g., a display, keyboard, and cursor control device may also be coupled to the bus 720 to support direct operator interaction with the computer system 700. Other operator and administrative interfaces can be provided through network connections connected through the communication port(s) 760. Components described above are meant only to exemplify various possibilities. In no way should the aforementioned exemplary computer system 700 limit the scope of the present disclosure.

While considerable emphasis has been placed herein on the preferred embodiments, it will be appreciated that many embodiments can be made and that many changes can be made in the preferred embodiments without departing from the principles of the disclosure. These and other changes in the preferred embodiments of the disclosure will be apparent to those skilled in the art from the disclosure herein, whereby it is to be distinctly understood that the foregoing descriptive matter is to be implemented merely as illustrative of the disclosure and not as a limitation.

### Advantages of the Invention

The present disclosure provides a system and a method to perform an Electroencephalography (EEG) diagnostics and a prognosis measurement.

The present disclosure facilitates analyzing wavelength height frequencies for certain disorder types by utilizing EEG scans, signal topology of the EEG scan, and raw data.

The present disclosure facilitates sending signals through an EEG headset or an external non-implantable Brain-Computer-Interface (BCI).

The present disclosure facilitates sending signals by utilizing binaural beats within a 30 HZ range or less to the brain to follow a neural interfacing or EEG frequency range of wavelengths.

The present disclosure facilitates analyzing EEG scan data by using a mathematical proof for a base data of EEG parameters of a healthy individual versus EEG parameters of a patient.

We claim:

1. A system for performing an Electroencephalography (EEG) diagnostics and a prognosis measurement, the system comprising:
one or more processors; and
a memory operatively coupled to the one or more processors, wherein the memory comprises processor-executable instructions, which on execution, cause the one or more processors to:
determine one or more parameters of an EEG frequency waveform captured during the EEG diagnostics;
evaluate the determined one or more parameters of the EEG frequency waveform prior to applying binaural beats or acoustic amplification to the EEG frequency waveform; and
compare the evaluated one or more parameters of the EEG frequency waveform and one or more parameters of a frequency waveform corresponding to a correlating diagnosis signal to perform the prognosis measurement, where the comparison is performed using a core metric.

2. The system as claimed in claim 1, wherein the system uses a pre-determined internal signal topology to perform the prognosis measurement and recommend a remedial treatment.

3. The system as claimed in claim 1, wherein the evaluated one or more parameters of the EEG frequency waveform are any or a combination of height, length, and frequency of the EEG frequency waveform, and the one or more parameters of the frequency waveform corresponding to the correlating diagnosis signal are any or a combination of height, length, and frequency of the waveform corresponding to the correlating diagnosis signal.

4. The system as claimed in claim 3, wherein the core metric is computed based on any or a combination of the height of the EEG frequency waveform, a minimal height of the frequency waveform of the correlating diagnosis signal, an endpoint of the frequency of the EEG frequency waveform, and a top point of the frequency of the EEG frequency waveform.

5. The system as claimed in claim 4, wherein a base of the height of the EEG frequency waveform and the minimal height of the frequency waveform of the correlating diagnosis signal are horizontally scalable.

6. The system as claimed in claim 1, wherein the EEG diagnostics is performed using an EEG headset and an external non-invasive Brain-Computer Interface (BCI), and the prognosis measurement is performed using one or more deep brain simulation mechanisms.

7. The system as claimed in claim 6, wherein the EEG headset is any of a dry node EEG headset and a wet node EEG headset.

8. The system as claimed in claim 1, wherein the EEG diagnostics and the prognosis measurement are performed in real-time without data latency and signal sensitivity.

9. The system as claimed in claim 1, wherein each of the EEG frequency waveform and the waveform corresponding to the correlating diagnosis signal is simulated within an EEG simulator to be used in clinical trials.

10. The system as claimed in claim 1, wherein the EEG frequency waveform is implemented in any of an EEG power spectrum, one or more spectral frequency ranges, a spectrum analysis, a magnitudal activity, and an awake/non awake EEG spectral, neuronal and density.

11. The system as claimed in claim 1, wherein a modular form of Binaural Beat Therapy (BBT) is derived from the EEG frequency waveform, and where the BBT is used to determine one or more base data points to perform a therapeutic neurostimulation.

12. The system as claimed in claim 1, wherein an Exposure and Response Prevention (ERP) therapy is used for the prognosis measurement without triggering recessive brain or neural patterns.

13. The system as claimed in claim 1, wherein a geometric topology is applied into a wave signal analysis in relation to raw EEG data along with one or more signal readings.

14. The system as claimed in claim 1, wherein the one or more parameters of the EEG frequency waveform of the diagnosis signal are modulated, triggered, and oscillated to perform the EEG diagnostics.

15. A method for performing an Electroencephalography (EEG) diagnostics and a prognosis measurement, the method comprising:
determining, by one or more processors, one or more parameters of an EEG frequency waveform captured during the EEG diagnostics;
evaluating, by one or more processors, the determined one or more parameters of the EEG frequency waveform prior to applying binaural beats or acoustic amplification to the EEG frequency waveform; and
comparing, by one or more processors, the evaluated one or more parameters of the EEG frequency waveform and one or more parameters of a frequency waveform corresponding to a correlating diagnosis signal to perform the prognosis measurement, where the comparison is performed using a core metric.

16. The method as claimed in claim 15, wherein the method comprises:
using a pre-determined internal signal topology to perform the prognosis measurement and recommend a remedial treatment.

17. The method as claimed in claim 15, wherein the evaluated one or more parameters of the EEG frequency waveform are any or a combination of height, length, and frequency of the EEG frequency waveform, and the one or more parameters of the frequency waveform corresponding to the correlating diagnosis signal are any or a combination of height, length, and frequency of the waveform corresponding to the correlating diagnosis signal.

18. The method as claimed in claim 17, wherein the core metric is computed based on any or a combination of the height of the EEG frequency waveform, a minimal height of the frequency waveform of the correlating diagnosis signal, an endpoint of the frequency of the EEG frequency waveform, and a top point of the frequency of the EEG frequency waveform.

19. The method as claimed in claim 18, wherein a base of the height of the EEG frequency waveform and the minimal height of the frequency waveform of the correlating diagnosis signal are horizontally scalable.

20. The method as claimed in claim 15, wherein the EEG diagnostics and the prognosis measurement are performed in real-time without data latency and signal sensitivity.
